(19) United States
(12) Patent Application Publication
(10) Pub. No.: US 2025/0041082 A1
(43) Pub. Date: Feb. 6, 2025

(54) INTERCHANGEABLE DISTAL END FOR A PROSTHETIC SOCKET SYSTEM

(71) Applicant: Ossur Iceland ehf, Reykjavik (IS)

(72) Inventors: Marco STEINBERG, Reykjavik (IS); Sigurdur HANNESSON, Reykjavik (IS); Andrew BACHE, Reykjavik (IS)

(21) Appl. No.: 18/924,599

(22) Filed: Oct. 23, 2024

Related U.S. Application Data
(63) Continuation of application No. 18/194,770, filed on Apr. 3, 2023, now Pat. No. 12,150,869.
Application No. 18/194,770 is a continuation of application No. 17/462,508, filed on Aug. 31, 2021, now Pat. No. 11,642,233.
(60) Provisional application No. 63/074,948, filed on Sep. 4, 2020.

(51) Int. Cl. A61F 2/78; A61F 2/50; A61F 2/66; A61F 2/80

(52) U.S. Cl. CPC A61F 2/7812; A61F 2/80; A61F 2002/5007; A61F 2002/5083; A61F 2002/6614

(57) ABSTRACT

A prosthetic socket system having an interchangeable distal end, includes a prosthetic socket defining a substantially rigid base, and a funnel formed from a polymeric material and removably attached to the base of a prosthetic socket. The funnel is arranged to be positioned in the base, and is configured and dimensioned to accommodate a distal end of a residual limb.

### CROSS-REFERENCE TO RELATED APPLICATIONS

This application is a continuation of U.S. patent application Ser. No. 18/194,770, filed Apr. 3, 2023, which is a continuation of U.S. patent application Ser. No. 17/462,508, filed Aug. 31, 2021, now U.S. Pat. No. 11,642,233, which claims benefit of U.S. Provisional Application No. 63/074,948, filed Sep. 4, 2020. This application also relates to U.S. Patent Application Publications 2018/0303637, published Oct. 25, 2018, 2018/0333279, published Nov. 22, 2018, and 2020/0222211, published Jul. 16, 2020. Each of these patent application publications is incorporated by reference.

### FIELD OF THE DISCLOSURE

The disclosure relates to interchangeable distal ends for a prosthetic socket system.

### BACKGROUND

A typical prosthetic leg and foot include a socket, pylon, and foot. A socket is commonly referred to as the portion of a prosthesis that fits around and envelops a residual limb or stump, and to which prosthetic components, such as a foot, are attached. The socket's fitting and alignment are difficult tasks to perform and require extensive knowledge, training, and skill for the prosthetist.

The socket must fit closely to the stump to provide a firm connection and support but must also be sufficiently loose to allow for circulation. In combination with proper fitting, the socket must transfer loads from the residual limb to the ground comfortably.

Conventional sockets are rigid and generally have a generally uniform shape, which receives a large portion of the residual limb. These sockets are permanently formed to a static customized shape, meaning the socket does not account for shape and volume fluctuations of the residual limb. When there are shape and volume fluctuations, the fitting of the socket is impeded, with these sockets causing discomfort, pain, and soft tissue breakdown of the stump. Conventional sockets also are bulky and cumbersome to wear and may be difficult to don, making the residual limb uncomfortable when worn.

Some attempts have been made to develop adjustable sockets with individual components that can be varied in size and/or shape to account for volume and shape fluctuations of the residual limb. However, these adjustable sockets tend to have labor-intensive and complicated tightening systems for donning and doffing the socket, making their use difficult for patients with limited dexterity, cognition, and/or strength. The drawbacks can cause the unsafe and improper use of the socket, causing discomfort and even injury.

Proper fitting of the distal end of a residual limb to the socket leads to better securing of the socket to the residual limb; however, because residual limbs have varying sizes and shapes, it can be challenging to provide a universal fit.

Given the foregoing, there is a need for an adjustable socket system that overcomes the problems of existing sockets.

### SUMMARY

Embodiments of the disclosure comprise an interchangeable distal end of adjustable sockets or universally fitting sockets. The interchangeable distal end is adapted to fit a variety of residual limb anatomies and may be easily adapted from the socket according to the demands incurred by the residual limb anatomies. Such demands may include sizing, shapes, fitting, and comfort.

As defined by this disclosure, the interchangeable distal ends generally refer to the combination of the socket and a funnel or insert placeable within a cavity of a prosthetic socket and located at the interior distal end. The funnel is adapted with a generally conical or partial conical shape or another suitable shape, approximating a general configuration or shape of a corresponding distal end.

The funnel intends to provide or embrace a set of differently shaped funnels configured to receive a distal end of a residual limb. The funnels may fill in voids within a distal end of the socket cavity and provide a more intimate fit between the socket and the residual limb. This arrangement is particularly beneficial when the socket is of the adjustable type that is adaptable in size according to an individual residual limb's specific sizing and shape. However, such funnels may be adapted to fit within a custom-formed prosthetic socket in the event the residual limb, particularly at the distal end, undergoes dimensional changes, and greater stability is required within the socket by which the funnel may be used and installed to take up space and provide a more intimate fit between the socket and the residual limb.

The funnel may include mounting means by which the funnel can be easily removed from the socket and swapped with another funnel better adapted to an individually shaped residual limb. The mounting means may be provided to offer more secure mounting or simplified mounting, such that the user may have ready means to change the funnels without the aid of a clinician. On the contrary, the mounting means may be sufficiently robust to require only a clinician to replace the funnel.

The funnel may be offered in various shapes, sizes, and properties to accommodate an individual user of a socket better. Such variations of the funnel may include its height relative to a mounting location of the socket, such as the extent by which it extends proximally from a distal end of the socket. Another variation may be a maximum diameter by which the funnel radially extends toward sidewalls or struts of the socket. Another variation may be the shape of the funnel, which may be configured to more intimately fit the shape of the socket or the shape of the residual limb. Yet another variation may be the material composition of the funnel, such as having softer or more rigid material properties to accommodate more sensitive areas of a residual limb. Further, another variation may be additional properties possessed by the funnel, such as being constructed from different materials at strategic locations to offer enhanced beneficial properties.

### BRIEF DESCRIPTION OF THE DRAWINGS

These and other features, aspects, and advantages of the present disclosure will better understand the following description, appended claims, and accompanying drawings.

FIG. 1 is an elevational view of an adjustable socket showing proximal and distal ends and relative directions of the adjustable socket.

FIG. 2A is a first perspective view showing an interchangeable distal end as a funnel in a simplified view of the adjustable socket of FIG. 1.

FIG. 2B is another perspective view of the funnel in the adjustable socket of FIG. 1.

FIG. 2C is a variation of the perspective view of the funnel in FIG. 2B, showing means for mounting the funnel to the base of the adjustable socket.

FIG. 2D is a variation of FIG. 2C with the base removed to show the means for mounting the funnel to the adjustable socket.

FIG. 3A is a top view of a variation of the funnel attached to the base of the adjustable socket.

FIG. 3B is a sectional view taken along line IIIB-IIIB in FIG. 3A.

FIG. 4 is a perspective view of the funnel in FIG. 2A.

FIG. 5 is a perspective view of a variation of the funnel in FIG. 4.

FIG. 6A is a perspective view of another embodiment of a funnel mounted in the base of FIG. 1.

FIG. 6B is a sectional view taken along line VIA-VIA in FIG. 6A.

FIG. 7A is a perspective view of another embodiment of a funnel mounted in the base of FIG. 1.

FIG. 7B is a sectional view taken along line VIA-VIA in FIG. 7A.

FIG. 8 is a schematic view of another embodiment of a funnel.

FIG. 9 is a schematic view of another embodiment of a funnel.

FIG. 10 is a perspective view of another embodiment of a funnel.

### DETAILED DESCRIPTION OF VARIOUS EMBODIMENTS

A better understanding of different embodiments of the disclosure may be had from the following description read with the accompanying drawings in which reference characters refer to like elements.

While the disclosure is susceptible to various modifications and alternative constructions, certain illustrative embodiments are in the drawings and are described below. It should be understood, however, there is no intention to limit the disclosure to the specific embodiments disclosed, but on the contrary, the intention covers all modifications, alternative constructions, combinations, and equivalents falling within the spirit and scope of the disclosure.

It will be understood that unless a term is expressly defined in this application to possess a described meaning, there is no intent to limit the meaning of such term, either expressly or indirectly, beyond its plain or ordinary meaning.

Any element in a claim that does not explicitly state “means for” performing a specified function or “step for” performing a specific function is not to be interpreted as a “means” or “step” clause as specified in 35 U.S.C. section 112(f).

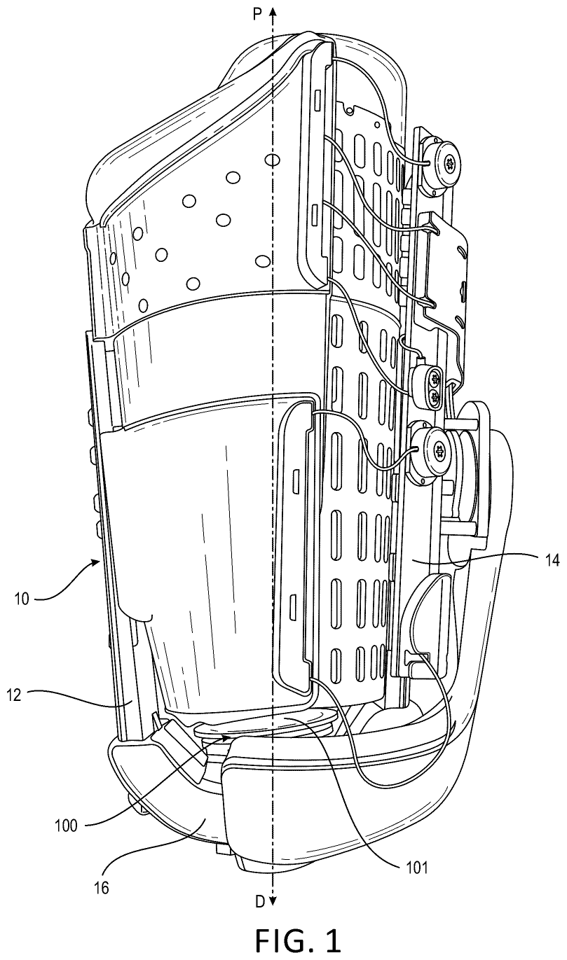

FIG. 1 displays an exemplary socket system 10 adapted to have an interchangeable distal end 100 according to embodiments of the disclosure. The socket system is disclosed in U.S. patent application publication 2018/0303637 and in U.S. patent application publications 2018/0333279 and 2020/0222211. However, the interchangeable distal end can likewise be provided in a conventional custom-made prosthetic socket. The interchangeable distal end comprises both components of the socket system and a removable funnel or set of funnels for attachment to a distal end D of the socket opposite a proximal end of the socket, and residing within a distal end of an internal cavity of the prosthetic socket.

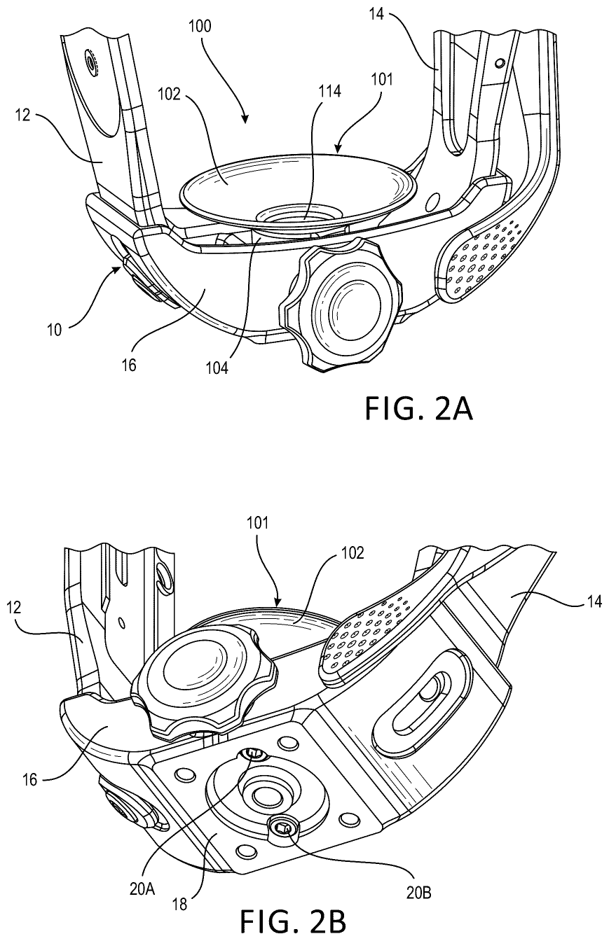

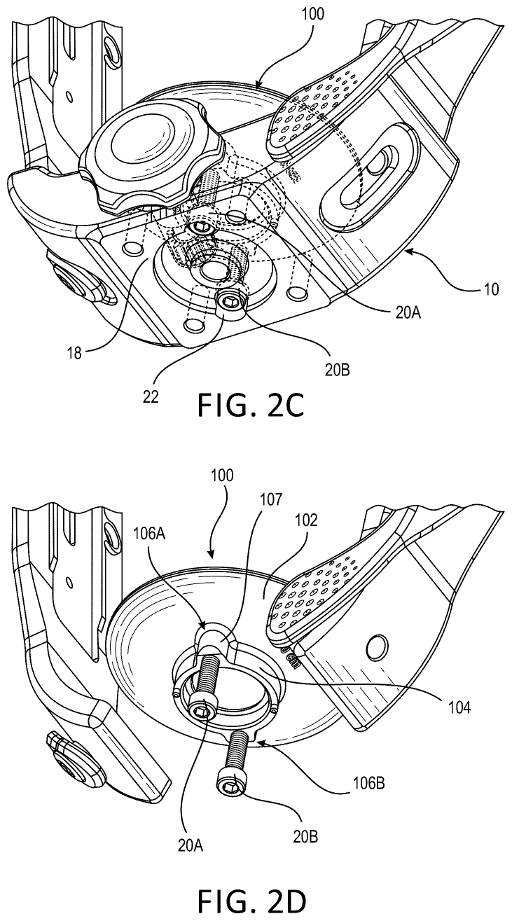

According to the exemplary socket system in FIGS. 1-3B, the interchangeable distal end 100 includes a funnel 101 removably attached to a base 16 of a prosthetic socket. The funnel 101 is positionable between first and second struts 12, 14 extend from the base 16. The funnel 101 forms a stem 104 extending from the receiving portion 102. In addition, the funnel 101 forms a central opening 114 extending through the receiving portion 102 and the stem 104, and is defined along an axis A-A of the funnel 101.

The base 16 defines a distal portion 18 and has mounting means for securing the base 16 to the stem 104 of the funnel 101. The mounting means 20, 22 comprises at least one threaded hole 22 for permitting insertion and retention of fasteners 20A, 20B adapted to engage the funnel 101.

The stem 104 of the funnel 101 may form at least one mounting portion 104A, 104B. The at least one mounting portion 106A, 106B is arranged to receive the at least one fastener 20A, 20B. The at least one mounting portion 106A, 106B forms a rib 107 protruding radially from the stem 104 and generally along the length of the stem 104. The rib 107 preferably defines an opening 116A, 116B, and a threaded periphery of the opening 116A, 116B is arranged to engage the at least one fastener 20A, 20B.

The receiving portion 102 of the funnel 101 defines a receiving surface 110 adapted to receive a distal end of a residual limb. A periphery 124 of the receiving portion 102 is generally circular. As shown, the receiving surface 110 is generally conical and concave to receive a distal end of a residual limb.

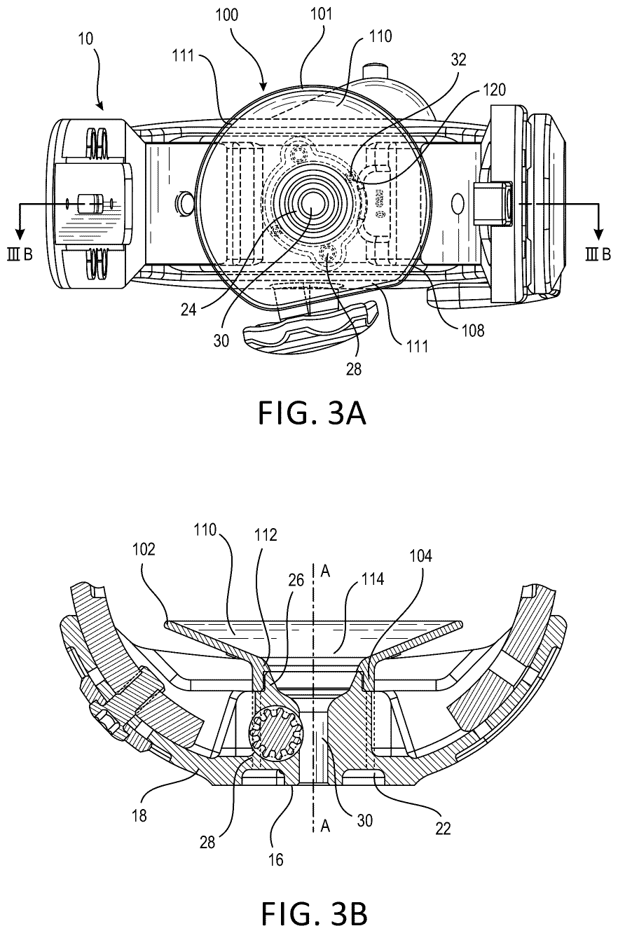

More generally, the receiving surface and the receiving portion may be arranged in any shape adapted to receive a distal end of a residual limb. For example, in FIG. 3A, the periphery 108 of the receiving portion 102 has a circular portion 109 and a linear portion 111. The linear portion 111 generally corresponds to the shape of the socket while offering a sufficient surface area to receive the residual limb.

Referring to FIG. 3B, the stem 104 defines an inner notch 112 formed about the opening 114. The inner notch 112 is adapted to rest along an edge of an inner ring 26 protruding from a proximal side of the base 16 about the central opening 114. The base 16 defines an elongate opening 30 corresponding to the axis A-A of the central opening 114. The elongate opening 30 preferably permitting insertion of a locking pin, as described in U.S. 2020/0222211, therethrough. In addition, the base 16 defines elongate openings 28 adapted to correspond to the at least one threaded hole 116A, 116B of the at funnel 101.

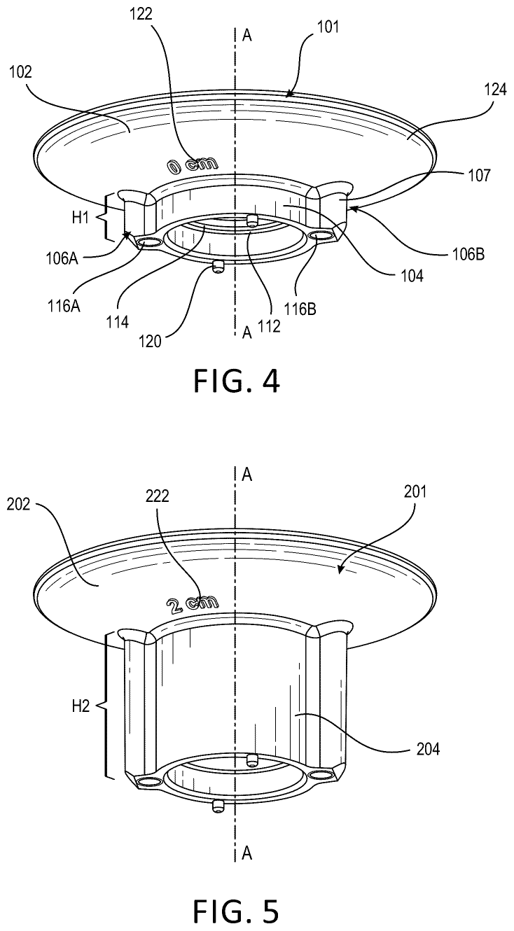

As best understood from FIGS. 3A and 4, the base 16 may define at least one aperture 32 along a proximal side of the base 16. The at least one aperture 32 is arranged to receive a protrusion 120 extending from a distal side of the funnel 101. This arrangement offers additional securing of the funnel to the base and rotational control relative to the base.

FIG. 4 shows a detailed view of the funnel 101. The funnel had a central axis A-A, and the central opening 114 extends through the central axis A-A. The receiving portion 102 is located about the central axis A-A and along a proximal portion of the funnel 101. The stem 104 extends distally from the receiving portion 102 and about the central axis A-A. As noted above, the receiving portion 102 has a generally conical shape and is concave in the distal direction to receive a distal end of a residual limb.

As in any of the disclosure embodiments, the funnel 101 preferably is formed from a polymeric material, such as an elastomer, to provide flexibility and toughness. Alternatively, the funnel 101 may be formed from a rigid polymeric material. The funnel 101 may include indicia 122 is provided on the funnel to show the height or size of the funnel 101.

Between FIGS. 4 and 5, a set of funnels may comprise the first funnel 101 having a stem 102 defining a first height H1, and a second funnel 201 having a stem 204 defining a second height H2. The first height H1 may differ from the second height H2. The different funnels may be distinguished based on other properties: height, diameter or shape of periphery of the receiving portion, or different material properties such as flexibility, compressibility, or rigidity.

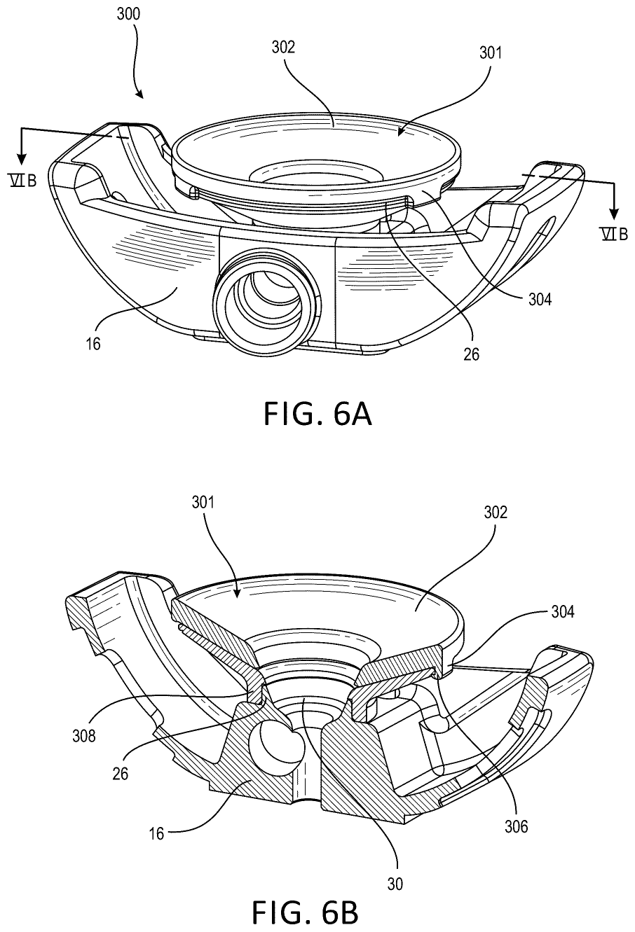

Referring to FIGS. 6A and 6B, another embodiment of an interchangeable distal end 300 includes a funnel 301 including a receiving portion 302 defining at least one tab 304 generally protruding axially from the receiving portion 302 and being adapted to secure to an annular insert 308 formed to attach to the base 16. The at least one tab 304 defines a hook 306 engageable with an edge of the annular insert 308. The funnel 301 may be formed from a resilient material, and the at least one tab 304 may be adapted to resiliently secure to the annular insert 308. In addition, the annular insert 308 may be adapted to secure to the inner ring 26.

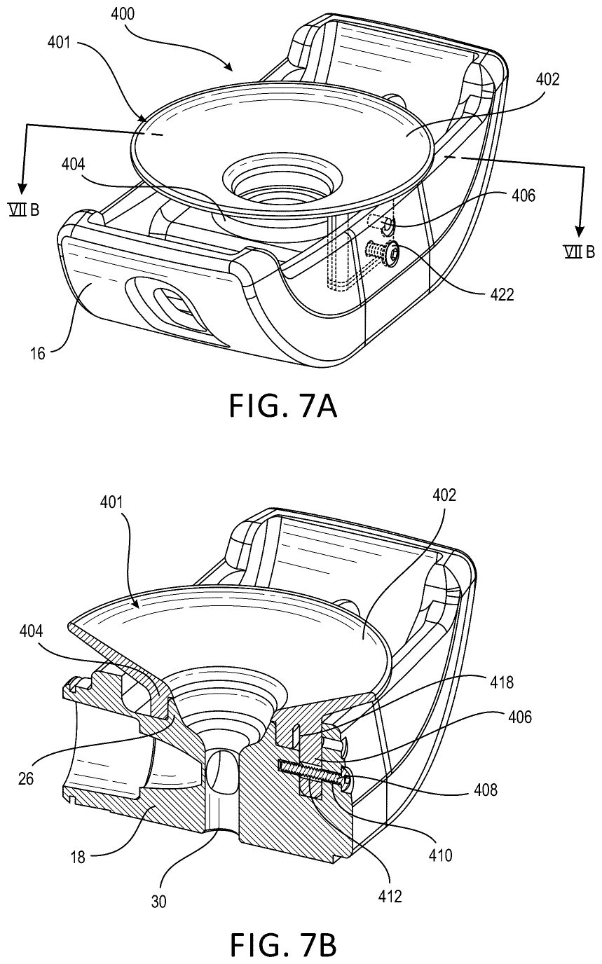

FIGS. 7A and 7B depict another embodiment of an interchangeable distal end 400 for a prosthetic socket. The interchangeable distal end 400 includes a funnel 401 defining a receiving portion 402 and a stem 404 protruding from the receiving portion 402. The funnel 401 includes a flange 406 extending from the receiving portion 402 and spaced apart a distance 418 from the stem 404. The stem 404 extends about an annular ring 26 of the base 16. The base 16 defines an opening 410 through which a fastener 408 and preferably laterally (generally perpendicularly to the axis of the funnel) extends through an opening 412 defined by the flange 406 and secures to the base 16.

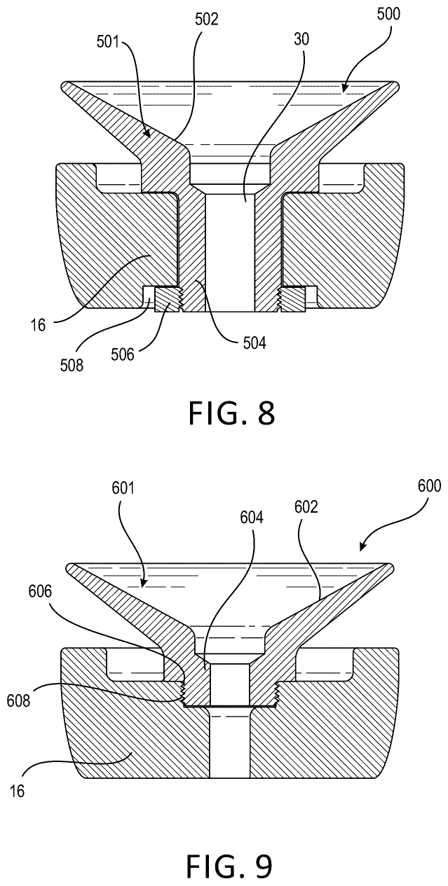

FIG. 8 illustrates another interchangeable distal end 500 for a prosthetic socket and includes a funnel 501 defining a receiving portion 502 and a stem 504 protruding from the receiving portion 502. A distal annular ring 506 secures the base 16 within a space 508 formed by the base 16.

FIG. 9 illustrates yet another interchangeable distal end 600 for a prosthetic socket and includes a funnel 601 defining a receiving portion 602 and a stem 604 protruding from the receiving portion 602. The stem 604 forms a threaded section 606 adapted to threadedly engage a threaded portion 608 defines by the base 16.

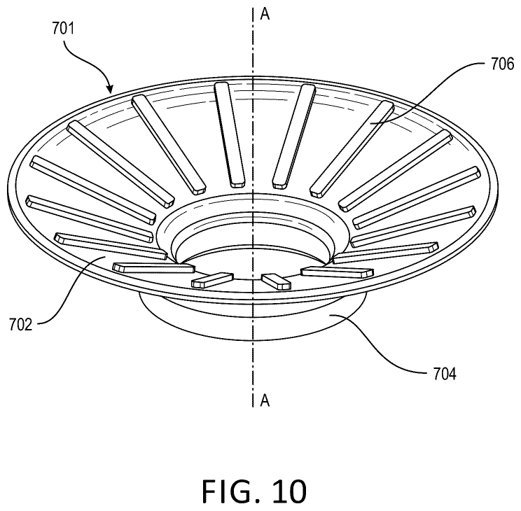

FIG. 10 exemplifies a funnel 701 having a receiving portion 702, including a surface 703 along which at least one feature 706 is secured. The at least one feature 706 is formed by a second material having different properties from a first material forming the receiving portion. The at least one feature 706 is formed as a plurality of strips radially extending from an axis A-A of the funnel 701. At least one feature 706 may be formed as any number of different shapes, sizes, and material compositions. The stem 704 may be devoid of such features or possess its own or similar features as the receiving portion 702.

Although this disclosure describes certain exemplary embodiments and examples of an interchangeable distal end, including a funnel, it nevertheless will be understood by those skilled in the art that the present disclosure extends beyond the specifically disclosed prosthetic socket and interchangeable distal end and funnel embodiments to other alternative embodiments and/or users of the disclosure and obvious modifications and equivalents thereof. It is intended that the scope of the present disclosure should not be limited by the particular disclosed embodiments described above, and may be extended to prosthetic devices and supports, and other applications that may employ the features described herein.

1. A prosthetic socket system having an interchangeable distal end, comprising:
a prosthetic socket defining a base;
a funnel formed from an elastomeric material and removably attached to the base of a prosthetic socket, the funnel arranged to be positioned in the base;
an annular insert formed to attach to the base;
wherein the funnel defines a receiving portion having a generally conical shape;
wherein the funnel forms a stem extending from the receiving portion;
wherein the funnel forms a central opening extending through the receiving portion and the stem, the central opening defined along a central axis of the funnel;
wherein the base defines an elongate opening co-axial to the central axis of the central opening;
wherein the funnel is more flexible than the base;
wherein the funnel defines at least one tab protruding axially from the receiving portion and being adapted to secure to the annular insert formed to attach to the base.

2. The prosthetic socket system of claim 1, wherein the at least one tab defines a hook engageable with an edge of the annular insert.

3. The prosthetic socket system of claim 1, wherein the funnel is formed from a resilient material.

4. The prosthetic socket system of claim 1, wherein the at least one tab is adapted to resiliently secure to the annular insert.

5. The prosthetic socket system of claim 1, wherein the annular insert may be adapted to secure to an inner ring protruding from a proximal side of the base about the central opening.

6. The prosthetic socket system of claim 1, wherein the base defines at least one elongate opening adapted to correspond to the opening of the funnel, and parallel to the central axis of the funnel.

7. The prosthetic socket system of claim 1, wherein the receiving portion defines a concave receiving surface configured and dimensioned to receive a distal end of a residual limb.

8. The prosthetic socket system of claim 1, wherein at least a portion periphery of the receiving portion is generally circular.

9. The prosthetic socket system of claim 1, wherein a periphery of the receiving portion has a circular portion and a linear portion, the linear portion corresponding to a shape of the prosthetic socket.

10. The prosthetic socket system of claim 1, wherein the receiving portion has a surface along which a plurality of strips radially extend from an axis A-A of the funnel.

11. A funnel configured and dimensioned for fitting in a base of a prosthetic socket, the funnel comprising:
a receiving portion having a generally conical shape;
a stem extending from the receiving portion, the funnel forming a central opening extending through the receiving portion and the stem, the central opening defined along a central axis of the funnel;
at least one mounting portion parallel to the central opening and disposed radially outwardly relative to the stem;
wherein the funnel is formed from an elastomeric material;
wherein the funnel defines at least one tab protruding axially from the receiving portion formed as a hook.

12. The funnel of claim 11, wherein the receiving portion defines a concave receiving surface configured and dimensioned to receive a distal end of a residual limb.

13. The funnel of claim 11, wherein a periphery of the receiving portion has a circular portion and a linear portion, the linear portion corresponding to a shape of the prosthetic socket.

14. The funnel of claim 11, wherein the base defines at least one elongate opening adapted to correspond to the opening of the funnel, and parallel to the central axis of the funnel.

15. The funnel of claim 11, wherein the receiving portion has a surface along which a plurality of strips radially extend from an axis A-A of the funnel.

16. A funnel configured and dimensioned for fitting in a base of a prosthetic socket, the funnel comprising:
a receiving portion having a generally conical shape;
a stem extending from the receiving portion, the funnel forming a central opening extending through the receiving portion and the stem, the central opening defined along a central axis of the funnel;
at least one mounting portion parallel to the central opening and disposed radially outwardly relative to the stem;
wherein the funnel is formed from an elastomeric material;
wherein the funnel includes a flange extending from the receiving portion and spaced apart a distance from the stem, the flange defining an opening defined by the flange and arranged for receiving a fastener.

17. The funnel of claim 16, wherein the receiving portion defines a concave receiving surface configured and dimensioned to receive a distal end of a residual limb.

18. The funnel of claim 16, wherein a periphery of the receiving portion has a circular portion and a linear portion, the linear portion corresponding to a shape of the prosthetic socket.

19. The funnel of claim 16, wherein the base defines at least one elongate opening adapted to correspond to the opening of the funnel, and parallel to the central axis of the funnel.

20. The funnel of claim 16, wherein the receiving portion has a surface along which a plurality of strips radially extend from an axis A-A of the funnel.
